(19) United States
(12) Patent Application Publication
(10) Pub. No.: US 2015/0166562 A1
(43) Pub. Date: Jun. 18, 2015

(54) METHODS AND PRODUCTS RELATED TO PROTOZOAN DISEASE

(71) Applicant: Massachusetts institute of Technology, Cambridge, MA (US)

(72) Inventors: Gregory P. Fournier, Somerville, MA (US); Eric Alm, Medford, MA (US)

(73) Assignee: Massachusetts institute of Technology, Cambridge, MA (US)

(21) Appl. No.: 14/414,557

(22) PCT Filed: Jul. 12, 2013
(86) PCT No.: PCT/US2013/050310

§ 371 Date: Jan. 13, 2015

Related U.S. Application Data
(60) Provisional application No. 61/671,596, filed on Jul. 13, 2012.

(51) Int. Cl. C07D 493/08; C07D 309/10; C07D 211/94

(52) U.S. Cl. CPC C07D 493/08; C07D 211/94; C07D 309/10

(57) ABSTRACT

The invention relates to methods and related products for treatment or protozoan disease. The invention also relates to new compositions of matter useful in the treatment of protozoan disease and methods of identifying additional active compounds.

### RELATED APPLICATIONS

This application claims priority under 35 U.S.C. §119(e) to U.S. Provisional Application Ser. No. 61/671,596, entitled “METHODS AND PRODUCTS RELATED TO PROTOZOAN DISEASE” filed on Jul. 13, 2012, which is herein incorporated by reference in its entirety.

### FEDERALLY SPONSORED RESEARCH

This invention was made with government support under Grant No. NNH06CC03B awarded by NASA. The government has certain rights in this invention.

### BACKGROUND OF INVENTION

Infectious diseases caused by protozoa are among the leading causes of death throughout the world. Alone, the protozoa from genus plasmodium—cause of malaria—is considered endemic in more than 100 countries and is the leading cause of death for children. Control efforts generally involve a combination of prevention and treatment methods. To some success, these measures have become standardized as the global effort to defeat malaria has become an international priority. However, effective treatments for malaria, and a variety of other protozoan diseases remain a challenge in the face of emerging drug resistance with a growing incidence within developing nations.

### SUMMARY OF INVENTION

In some aspects the invention is a method for treating protozoan diseases by administering to a subject having a protozoan disease an inhibitor of protozoan tyrosyl-tRNA synthetase in an effective amount to treat the protozoan disease, wherein the inhibitor of protozoan tyrosyl-tRNA synthetase does not inhibit mammalian tyrosyl-tRNA synthetase. In some embodiments the inhibitor of protozoan tyrosyl-tRNA synthetase specifically binds to an adenylate binding pocket of protozoan tyrosyl-tRNA synthetase. In other embodiments the adenylate binding pocket is a region of the protozoan tyrosyl-tRNA synthetase corresponding to F70, Q80, K84, C85 and F127 of SEQ ID NO. 1. In another embodiment, the adenylate binding pocket is a region of the protozoan tyrosyl-tRNA synthetase corresponding to F70, Q80, and F127 of SEQ ID NO. 1.

In some embodiments of this invention, the inhibitor of protozoan tyrosyl-tRNA synthetase is also an inhibitor of bacterial tyrosyl-tRNA synthetase. In other embodiments, the inhibitor of protozoan tyrosyl-tRNA synthetase is not an inhibitor of bacterial tyrosyl-tRNA synthetase.

In some embodiments of the invention, the protozoan diseases treated include but are not limited to selection from groups including malaria, toxoplasma, leishmaniasis, giardiasis, trypanosomiasis (Sleeping sickness, Chagas disease), amoebic dysentery, and trichomoniasis.

In an embodiment of the invention, an inhibitor of protozoan tyrosyl-tRNA synthetase includes but is not limited to the compound of Formula (I)

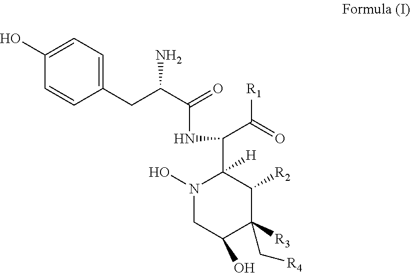

or a salt thereof;

wherein:

each instance of R₁, R₂, R₃, and R₄ is, independently, hydrogen, hydroxyl, substituted or unsubstituted OR′, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; R′ is substituted or unsubstituted alkyl, substituted or unsubstituted alkenyl, substituted or unsubstituted alkynyl.

In a further embodiment of the invention, the inhibitors of protozoan tyrosyl-tRNA synthetase include but are not limited to the following: SB 219383, SB 239629, SB 243545, or SB 284485.

Another aspect of the invention involves a composition, comprising: an inhibitor of protozoan tyrosyl-tRNA synthetase, wherein the inhibitor of protozoan tyrosyl-tRNA synthetase does not inhibit mammalian or bacterial tyrosyl-tRNA synthetase.

Yet another aspect of this invention involves a method of selecting or designing a compound that interacts with a protozoan tyrosyl-tRNA synthetase and modulates protozoan tyrosyl-tRNA synthetase activity, the method comprising the step of assessing the stereochemical complementarity between the compound and a topographic region of protozoan tyrosyl-tRNA synthetase, wherein the topographic region of the protozoan tyrosyl-tRNA synthetase is characterized by at least a portion of the amino acids positioned at an adenylate binding pocket of the protozoan tyrosyl-tRNA synthetase. In an embodiment of this invention, the adenylate binding pocket is a region of the protozoan tyrosyl-tRNA synthetase corresponding to F70, Q80, K84, C85 and F127 of SEQ ID NO. 1. In a further embodiment of the invention, the adenylate binding pocket is a region of the protozoan tyrosyl-tRNA synthetase corresponding to F70, Q80, and F127 of SEQ ID NO. 1.

Another aspect of the invention involves a composition comprising an inhibitor of protozoan tyrosyl-tRNA synthetase and an anti-protozoan agent. In an embodiment of this invention, the inhibitor of protozoan tyrosyl-tRNA synthetase synthetase is a compound of Formula (I):

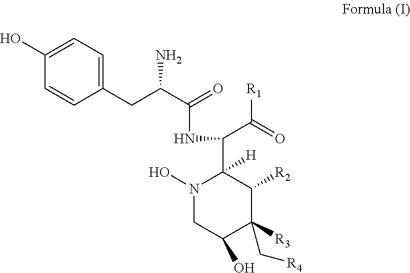

wherein the inhibitor of protozoan tyrosyl-tRNA or a salt thereof; wherein: each instance of R₁, R₂, R₃, and R₄ is, independently, hydrogen, hydroxyl, substituted or unsubstituted OR′, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; R′ is substituted or unsubstituted alkyl, substituted or unsubstituted alkenyl, substituted or unsubstituted alkynyl. In another embodiment of the invention, where the composition comprises an inhibitor of protozoan tyrosyl-tRNA synthetase and an anti-protozoan agent, the inhibitor of protozoan tyrosyl-tRNA synthetase includes but is not limited to SB 219383, SB 239629, SB 243545, and SB 284485.

In another aspect of the invention, the compound is the formula (I) or (II):

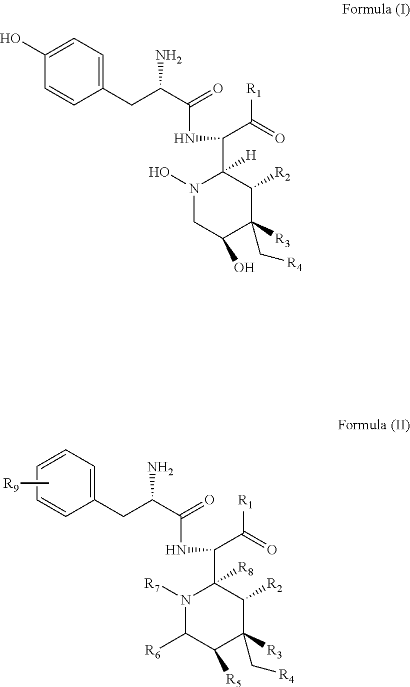

or a salt thereof; wherein:

each instance of R₁, R₂, R₃, and R₄, is independently, hydrogen, hydroxyl, substituted or unsubstituted OR′, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; R′ is substituted or unsubstituted alkyl, substituted or unsubstituted alkenyl, substituted or unsubstituted alkynyl; wherein each instance of R₅, R₆, R₇, R₈ and R₉ is, independently, hydrogen, halogen, substituted or unsubstituted alkyl, substituted or unsubstituted alkenyl, substituted or unsubstituted alkynyl; and provided that the compound of Formula (I) is not a compound wherein R₁ is a butyl ester or OH and R₂, R₃, and R₄ are all OH, provided that the compound of Formula (II) is not a compound wherein R₁ is a butyl ester or OH, and R₂, R₃, R₄, R₅, and R₇ are all OH, R₉ are all H and one OH, and R₆ and R₈ are both H.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows multiple sequence alignments (SEQ ID NOs. 6-13) of the region of TyrRS corresponding to the unique components of the adenylate binding pocket in protozoa, with 5 unique sites labeled with a box.

FIG. 2 shows a chemical structure of SB243545 with important protozoan sites identified.

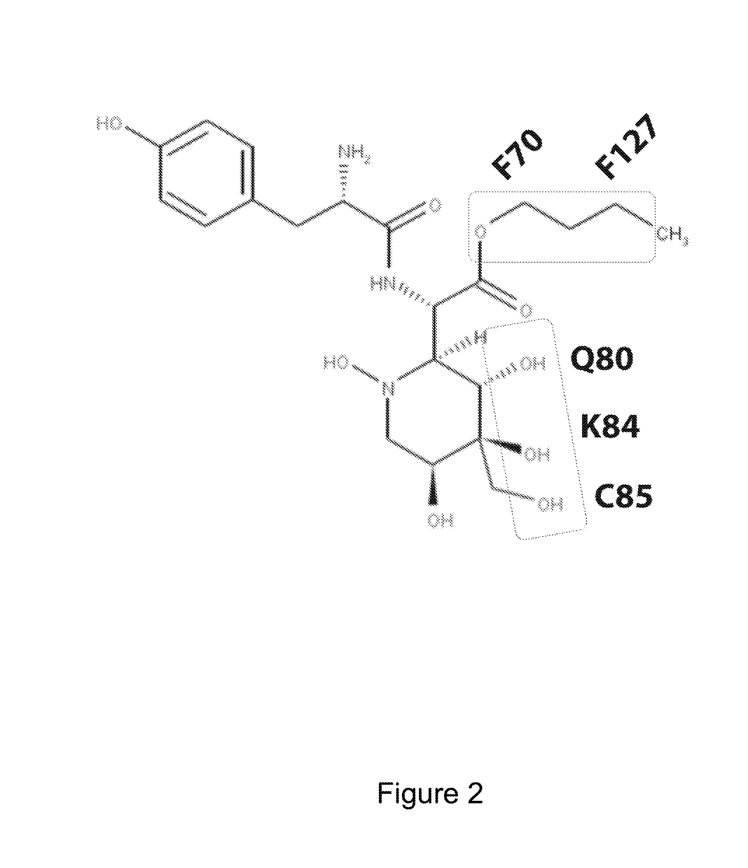

### DETAILED DESCRIPTION

A major challenge in the treatment of protozoan diseases is that most effective protein targets are highly conserved, and thus too similar across eukaryotes. As a result many potentially effective compounds would target both parasite and host. The discovery, according to the invention, of a horizontal gene transfer from a more distantly related organism, circumvents current limitations by providing a target, thus greatly increasing the potential selectivity of compounds targeted to protozoa. Horizontal gene transfer refers to the transfer of genetic material between organisms. As opposed to the vertical transfer of genetic information which occurs between parent and offspring generations, horizontal gene transfer is an exchange of genetic material with a different population or species that occurs independently of reproduction, and is subsequently inherited by offspring generations.

Many effective protein targets in protozoans are highly conserved, and thus are too similar to proteins in other non-protozoan eukaryotes, including humans and animals. Therefore, many effective protein targets in protozoans are highly conserved, and are too similar across eukaryotes. The discovery that protozoan tyrosyl-tRNA synthetase, a critical protein to protozoan survival, has distinct amino acids in important binding regions has vast implications for the development of protozoan therapeutics. The tyrosyl-tRNA synthetase target is particularly valuable as it is highly expressed, universally essential, and of a class often effectively targeted by antibiotics within bacteria.

Thus, the invention involves, in some aspects, methods for treating protozoan disease. The therapeutic methods of the invention are accomplished using an inhibitor of protozoan tyrosyl-tRNA synthetase. Tyrosyl-tRNA synthetase is an enzyme that is found in all cells. It plays an important role in the synthesis of proteins. During protein synthesis, amino acids are joined and specifically ordered to create proteins. Specifically the amino acid tyrosine is added at the proper place in a protein's chain of amino acids as a function of the tyrosyl-tRNA synthetase activity.

The crystal structure of Staphylococcus aureus tyrosyl-tRNA synthetase is disclosed in Qiu et al., Protein Science, 2001, v. 10, p. 2008. Based on this crystal structure combined with the knowledge of the critical amino acid differences in the protozoan tyrosyl-tRNA synthetases based on the teachings of the instant disclosure it is now possible to design therapeutic compounds that specifically inhibit protozoan tyrosyl-tRNA synthetase.

An “inhibitor of protozoan tyrosyl-tRNA synthetase” as used herein refers to any compound that reduces to any extent, partially or fully or completely inhibits the activity of protozoan tyrosyl-tRNA synthetase. The level of activity of protozoan tyrosyl-tRNA synthetase may be assessed using known in vitro or in vivo assays. In some embodiments the level of protozoan tyrosyl-tRNA synthetase activity may be reduced by 10%, 20%, 30%, 40%, 50%, 55%, 60%, 65%, 70%, 75%, 80%, 85%, 90%, 95%, 96%, 97%, 98%, 99% or greater. In some embodiments the level of protozoan tyrosyl-tRNA synthetase activity may be reduced by 100%.

Compounds useful as inhibitors of the invention may specifically inhibit protozoan tyrosyl-tRNA synthetase. A compound that specifically inhibits protozoan tyrosyl-tRNA synthetase is a compound that reduces to any degree or inhibits protozoan tyrosyl-tRNA synthetase, but that does not therapeutically impact or inhibit human or animal or mammalian tyrosyl-tRNA synthetase. The phrase “therapeutically impact or inhibit human or animal or mammalian tyrosyl-tRNA synthetase” refers to a level of tyrosyl-tRNA synthetase activity.

In some embodiments a compound is said to not therapeutically impact human or animal tyrosyl-tRNA synthetase in an in vitro assay of human or animal tyrosyl-tRNA synthetase activity. In that assay the compound may inhibit activity of the human or animal tyrosyl-tRNA synthetase by less than 50%, 40%, 35%, 30%, 25%, 20%, 15%, 10%, 5%, 4%, 3%, 2%, 1%, or 0.5%.

In some embodiments it is desirable for a compound that specifically inhibits protozoan tyrosyl-tRNA synthetase to be a compound that reduces to any degree or inhibits protozoan and bacterial tyrosyl-tRNA synthetase. In other embodiments it is desirable for a compound that specifically inhibits protozoan tyrosyl-tRNA synthetase to be a compound that reduces to any degree or inhibits protozoan tyrosyl-tRNA synthetase, but that does not therapeutically impact bacterial tyrosyl-tRNA synthetase. The phrase “therapeutically impact bacterial tyrosyl-tRNA synthetase” refers to a level of bacterial tyrosyl-tRNA synthetase activity.

In some embodiments a compound is said to not therapeutically impact bacterial tyrosyl-tRNA synthetase in an in vitro assay of bacterial tyrosyl-tRNA synthetase activity. In that assay the compound may inhibit activity of the bacterial tyrosyl-tRNA synthetase by less than 50%, 40%, 35%, 30%, 25%, 20%, 15%, 10%, 5%, 4%, 3%, 2%, 1%, or 0.5%.

The inhibitor of protozoan tyrosyl-tRNA synthetase may specifically interact with or near an adenylate binding pocket of protozoan tyrosyl-tRNA synthetase. The adenylate binding pocket of protozoan tyrosyl-tRNA synthetase is known in the art. It has been discovered herein that the protozoan and animal tyrosyl-tRNA synthetases have important amino acid distinctions, particularly within this adenylate binding pocket, that create the opportunity for the development of drugs that bind to and disrupt activity of the protozoan tyrosyl-tRNA synthetase without significantly affecting the animal tyrosyl-tRNA synthetase. An exemplary amino acid sequence of a protozoan tyrosyl-tRNA synthetase is provided herein to highlight the adenylate binding pocket and the important amino acid distinctions. However, the invention is not limited to the exemplified amino acid sequence. Alternative sequences for this and other protozoan tyrosyl-tRNA synthetases are encompassed within the invention. The exemplary amino acid sequence of Plasmodium vivax is:

(SEQ ID NO. 1)
MEGEGVKREELEGATGEAAPQEAASQEATPQEDVSAKLADILSVASECI
QPEELKARLLLKRRLVCYDGFEPSGRMHIAQGLLKCQIVNKLTSNGCRF
IFWIADWFAQLNNKMSGDLKKIKKVGMYFIEVWKSCGMNMQNVEFLWAS
EEINKKPNEYWSLVIDISKSFNINRIKRCLKEVIGRSEGEENYCSQILY
PCMQCADIFFLNVDICQLGIDQRKVNMLAREYCEIKKMKKKPIILSHQM
LPGLLEGQEKMSKSDENSAIFMDDSEADVNRKIKKGYCPPGVIESNPIF
AYARSIVFPHYNEFALQRKEKNGGNKTYATIAELEADYLSGALHPLDLK
DNVAIYLNKMLQPVRDHFQNDAAAKSLLSEIKKYKVTK

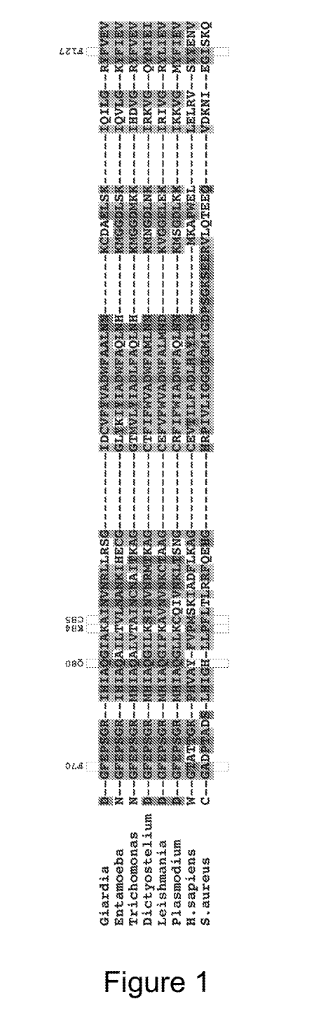

An amino acid Sequence Alignment is depicted in FIG. 1. Portions of SEQ ID NO. 1 are shown in FIG. 1 in the sixth line down identified as Plasmodium. Interesting residues of the Plasmodium sequence are compared against corresponding sequences of other protozoan tyrosyl-tRNA synthetases and a human tyrosyl-tRNA synthetase (H. sapiens) and a bacterial tyrosyl-tRNA synthetase (S. aureus). In particular, the amino acid positions corresponding to F70, Q80, K84, C85 and F127 of the Plasmodium vivax tyrosyl-tRNA synthetase in the depicted sequences are included in labeled boxes. These residues are in or near to the adenylate binding pocket. Inhibitors which specifically recognize one or more or all of these amino acids in a plasmodium and/or bacterial tyrosyl-tRNA synthetase may be useful in the methods of the invention.

The inhibitor of protozoan tyrosyl-tRNA synthetase in some aspects is a structural analog of tyrosyl-adenylate, the natural bound substrate of tyrosyl-tRNA synthetase protein (TyrRS). Structural analogs of tyrosyl-adenylate include, for example, naturally occurring tyrosyl-adenylate analog SB-219383, and synthesized derivatives SB-239629, SB-243545, and SB-284485. Other structural analogs include those described in publications such as Qiu et al., Protein Science, 2001, v. 10, p. 2008, Brown et al Bioorganic & Medicinal Chemistry Letters, 2001, v. 11, p. 711, and Jarvest et al Bioorganic & Medicinal Chemistry Letters, 2001, v. 11, p. 715.

Novel compounds for the inhibition of protozoan disease causing organisms are also provided. These compounds may be prepared by modifying known analogs such as SB-219383, SB-239629, SB-243545, and SB-284485 to provide improved selective and specific binding and inhibition of protozoan TyrRS. For instance the modifications may be such that they provide altered and preferably improved interactions at unique sites of protein interaction within and near the adenylate binding pocket, as defined in the Plasmodium vivax TyrS protein sequence, at sites F70, Q80, K84, C85, F127, as well as at adjacent structural sites. For example, chemical modifications of the analogous structure region of the butyl-ester group in SB-243545, may be made in order to accommodate the shape of the protozoan-specific binding pocket imposed by F70, Q80, and F127. Other alternative or supplemental chemical modifications of the 2′, 3′, and 4′ hydroxyl groups of the adenylate analog ring in all of the structural variants may be made, in order to specifically interact with the protozoan-specific amino acids at sites Q80, K84, and C85. Compound variants that are highly selective against Plasmodium, made be generated by making chemical modifications to the 3′ and 4′ hydroxyl groups on the adenylate ring, such that a strong interaction with a unique, reactive Cys residue at position 85 conserved within the Plasmodium genus is produced.

In some aspects of the invention the inhibitor of protozoan tyrosyl-tRNA synthetase is the compound of formula (I) or (II) or salts thereof.

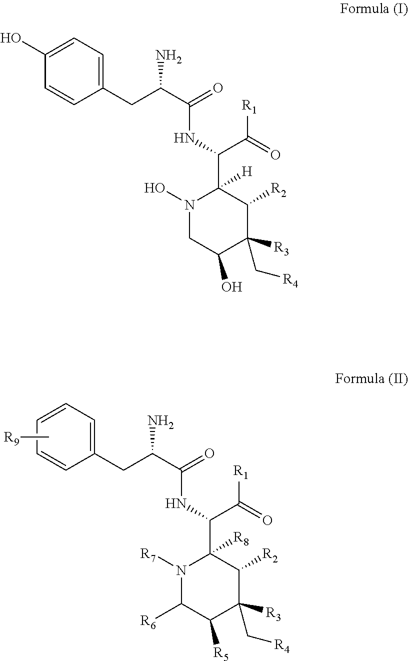

wherein each instance of R₁, R₂, R₃, and R₄ is, independently, hydrogen, hydroxyl, substituted or unsubstituted OR′, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

R′ is substituted or unsubstituted alkyl, substituted or unsubstituted alkenyl, substituted or unsubstituted alkynyl;

wherein each instance of R₅, R₆, R₇, R₈ and R₉ is, independently, hydrogen, halogen, substituted or unsubstituted alkyl, substituted or unsubstituted alkenyl, substituted or unsubstituted alkynyl; and

provided that the compound of Formula (I) is not a compound wherein R₁ is a butyl ester or OH and R₂, R₃, and R₄ are all OH,

provided that the compound of Formula (II) is not a compound wherein R₁ is a butyl ester or OH, and R₂, R₃, R₄, R₅, and R₇ are all OH, R₉ are all H and one OH, and R₆ and R₈ are both H.

In some embodiments the inhibitor of protozoan tyrosyl-tRNA synthetase is selected from the following compounds or salts thereof:

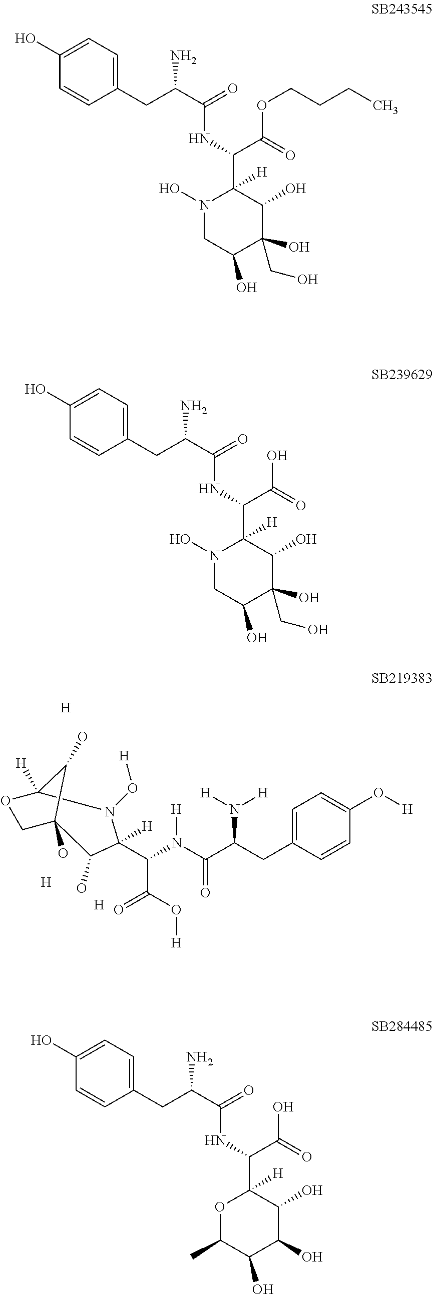

The invention also embraces analogs of the specific inhibitors described specifically or through incorporation by reference herein. Analogs are chemically modified versions of these inhibitors. In some embodiments, the inhibitor analogs have one or more activities of the specific inhibitors (as described herein), e.g. anti-protozoan activity. The one or more activities are preferably present in the inhibitor analogs in significant amounts, e.g., at greater than 10%, 20%, 30%, 40%, 50%, 60%, 70%, 80%, or 90% of the activity of the specific inhibitors. More preferably, the one or more activities are preferably present in the inhibitor analogs at greater than 100%, 110%, 120%, 130%, 140%, 150%, 160%, 170%, 180%, 190%, 200%, or more, of the activity of the specific inhibitors described herein. The inhibitor analogs may not have all of the activities of the specific inhibitors. However, non-active inhibitor analogs, having none of the anti-protozoan activities of specific inhibitors in significant amounts, are not useful in the methods of the invention.

According to the invention, useful inhibitors of protozoan tyrosyl-tRNA synthetase include any one or more inhibitors of protozoan tyrosyl-tRNA synthetase including but not limited to SB-219383, SB-239629, SB-243545, and SB-284485 and/or stereoisomeric forms, or pharmaceutically acceptable acid or base addition salt forms thereof or analogs thereof, in therapeutically effective amounts. The following publications describe compounds that are useful as inhibitors of protozoan tyrosyl-tRNA synthetase: Qiu et al., Protein Science, 2001, v. 10, p. 2008, Brown et al Bioorganic & Medicinal Chemistry Letters, 2001, v. 11, p. 711, and Jarvest et al Bioorganic & Medicinal Chemistry Letters, 2001, v. 11, p. 715. The disclosures of these publications are incorporated by reference herein in their entirety for the disclosure of inhibitors of tyrosyl-tRNA synthetase that are useful in the methods of the invention.

The invention embraces analogs of the specific inhibitors that, while identical in their chemical inhibition and binding of protozoan TyrRS, produce the additional activities of increased cell uptake/persistence, so as to effectively increase their binding and inhibition. For example, some modification of the compound may decrease the charge, allowing for passage through the cell membrane. The compounds are, thus, effective without increase binding affinity or specificity to the target.

These different inhibitor compounds and preparations described herein can be administered as a single dose or in several doses administered over a period of time (e.g. chronic administration at regular intervals of time) as described herein. Methods and compositions of the invention include stereoisomeric forms and pharmaceutically acceptable acid or base addition salt forms of the inhibitors of protozoan tyrosyl-tRNA synthetase.

Additionally, inhibitors of protozoan tyrosyl-tRNA synthetase include, for instance, inhibitory nucleic acids.

A protozoan tyrosyl-tRNA synthetase inhibitory nucleic acid causes specific gene knockdown, while avoiding off-target effects, such as knockdown of the human tyrosyl-tRNA synthetase. Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA or shRNA-mir expression constructs) are used to reduce expression of a gene (e.g., a protozoan tyrosyl-tRNA synthetase) in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a protozoan-specific promoter operably linked to a gene encoding the small interfering nucleic acid. In some cases, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluoyl nucleotides maintain gene silencing activity (Xia et al., ASC Chem. Biol. 1(3):176-83, (2006)). siRNAs with amide-linked oligoribonucleosides have been generated that are more resistant to S1 nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006). Other molecules that can be used to inhibit expression of a gene (e.g., a CSC-associated gene) include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat. Med. 4(8):967-71, 1998). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). The diverse array of suppression strategies that can be employed includes the use of DNA and/or RNA aptamers that can be selected to target a protein of interest (e.g, a protozoan tyrosyl-tRNA synthetase).

In one aspect, the invention provides methods for the treatment of protozoan disease. “Protozoan disease”, as used herein, refers to a condition resulting from infection by a host with a protozoan. A host, as used herein, refers to a subject. As used herein, a subject is a human or non-human animal such as non-human primates, dogs, cats, sheep, goats, cows, pigs, horses and rodents. Thus the invention encompasses the use of the inhibitors described herein alone or in combination with other therapeutics for the treatment of a subject having or at risk of having a protozoan infection. Typically, the subject that has had contact with a protozoan and the protozoan has invaded the body of the subject. The word “invade” as used herein refers to contact by the protozoan with an external surface of the subject, e.g., skin or mucosal membranes and/or refers to the penetration of the external surface of the subject by the protozoan, indirect protozoan penetration of the external surface of the subject by an infected insect, or ingestion of the protozoan via contaminated food or water. A subject at risk of having a protozoan infection is one that has been exposed to or may become exposed to a protozoan or a geographical area in which a protozoan infection has been reported. Further risks include close contact with a human or non-human primate or their tissues infected with the protozoan. Such persons include laboratory or quarantine facility workers who handle non-human primates that have been associated with the disease. In addition, hospital staff and family members who care for patients with the disease are at risk if they do not use proper barrier nursing techniques.

The methods of the invention are useful for treating a subject in need thereof. A subject in need thereof is a subject having or at risk of having a protozoan infection. In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms arising from the condition. If the subject in need of treatment is one who is at risk of having a condition, then treating the subject refers to reducing the risk of the subject having the condition or, in other words, decreasing the likelihood that the subject will develop an infectious disease to the virus, as well as to a treatment after the subject has been infected in order to fight the infectious disease, e.g., reduce or eliminate it altogether or prevent it from becoming worse.

The inhibitors described herein are isolated molecules. An isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation or be mixed with some of the components with which it is associated in nature, the molecular species may comprise only a small percentage by weight of the preparation. The molecular species is nonetheless substantially pure in that it has been substantially separated from the substances with which it may be associated in living systems.

Protozoan are typically microscopic, one-celled organisms that are heterotrophic (using organic carbon as a source of energy). They belong to any of the major lineages of protists and, are eukaryotes. As such they possess a “true,” or membrane-bound, nucleus. Many protozoan are symbionts of other organisms. They are able to multiply in humans, which contributes to their survival and also permits serious infections to develop from just a single organism. Some medically important protozoans include Archaezoa (Giardia lamblia, Trichomonas vaginalis), Amoebozoa (Entamoeba histolytica), Apicomplexa (Babesia microti, Cryptosporidium, Cyclospora, Plasmodium, Toxoplasma Gondii), Euglenozoa (Trypanosoma cruzi, T.b. gambiense, T.b. rhodesiense).

Examples of protozoan species include Leishmania aethiopica, Leishmania amazonensis, Leishmania arabica, Leishmania archibaldi, Leishmania aristedesi, Leishmania (Viannia) braziliensis, Leishmania chagasi (syn. Leishmania infantum), Leishmania (Viannia) colombiensis, Leishmania deanei, Leishmania donovani, Leishmania enriettii, Leishmania equatorensis, Leishmania forattinil, Leishmania garnhami, Leishmania gerbili, Leishmania (Viannia) guyanensis, Leishmania herreri, Leishmania hertigi, Leishmania infantum, Leishmania killicki, Leishmania (Viannia) lainsoni, Leishmania major, Leishmania mexicana, Leishmania (Viannia) naiffi, Leishmania (Viannia) panamensis, Leishmania (Viannia) peruviana, Leishmania (Viannia) pifanoi, Leishmania (Viannia) shawi, Leishmania tarentolae, Leishmania tropica, Leishmania turanica, Leishmania venezuelensis, Plasmodium berghei, Plasmodium brasilianum, Plasmodium chabaudi, Plasmodium cynomolgi, Plasmodium falciparum spp., Plasmodium gallinaceum, Plasmodium knowlesi, Plasmodium lophurae, Plasmodium malariae, Plasmodium ovale, Plasmodium relictum, Plasmodium vivax, Plasmodium yoelii, Trypanosoma avium, which causes trypanosomiasis in birds, T. boissoni, T. brucei, which causes sleeping sickness in humans and nagana in cattle, T. carassii, T. cruzi, which causes Chagas disease in humans, T. congolense, which causes nagana in cattle, horses, and camels, Trypanosoma equinum Voges 1901, T. equiperdum, which causes dourine or Covering sickness in horses and other Equidae, T. evansi, which causes one form of the disease surra in certain animals (human infection reported in 2005 in India), Trypanosoma lewisi, Trypanosoma melophagium, Trypanosoma percae, Trypanosoma rangeli, T. rotatorium in amphibian, T. simiae, which causes nagana in animals, T. suis, T. theileri, T. triglae in marine teleosts, T. vivax, which causes the disease nagana, Trichomonas vaginalis, Entamoeba histolytica, Giardia lamblia, Eimeria acervulina, Eimeria tenella, Eimeria maxima, and Sarcocystis species, particularly the species that infect humans, opossums, and horses.

The protozoan disease does not include disease associated with fungal or yeast infections or candida or worms.

Protozoan infections are widely known mainly around tropical or subtropical regions, and can be exemplified by malaria, leishmaniasis, African trypanosomiasis (African sleeping sickness), American trypanosomiasis (Chaga's disease), lymphatic filariasis, and babesiosis. These infections can be classified into those infecting only humans, and zoonosis also infecting domestic livestock or small animals, both leading to significant economic and social loss, as well as providing reservoirs for human disease, or as essential non-human vectors in a parasitic life cycle infecting humans at another stage. Thus, the methods of the invention include methods of treating humans as well as non-human animals.

Leishmaniasis is a life-threatening disease that is a major health problem worldwide. An estimated 10-15 million people are infected, and 400,000 new cases occur each year. Currently no vaccine is available against Leishmania and the generic, antimony-based drug treatments are plagued with low efficacy, high toxicity and widespread resistance.

Trypanosoma cause life-threatening diseases in humans, including African sleeping sickness and Chagas disease, as well as a number of important diseases in domestic animals. Leishmania and Trypanosoma are closely-related genera, representing the major pathogens in the kinetoplastid group of protozoa.

The intestinal parasite Entamoeba histolytica causes amoebic dysentery and extraintestinal abscesses of organs such as the liver and lung. The most commonly used drug for treating E. histolytica infection is metronidazole. Other free-living amoeba, which occasionally cause infections in humans, include Acanthamoeba and Naegleria spp.; these infections are typically difficult to treat.

Toxoplasma gondii; and several protozoans of veterinary importance such as Sarcocystis; Theileria; Babesia; and Eimeria (causing coccidiosis in fowl and domestic animals) are also the cause of relevant protozoan diseases.

Toxoplasma gondii is the causative agent in toxoplasmosis. In contrast to the mild clinical symptoms of infection seen in a healthy individual with an intact immune system, subjects with weakened, or otherwise compromised, immune systems can have serious clinical effects from toxoplasma infection. Toxoplasma gondii is also pathogenic to animals, particularly sheep, in which it causes abortion, stillbirth, and fetal mummification. In addition, Toxoplasma gondii causes encephalitis, a dangerous life-threatening disease in both man and domestic animals. Toxoplasma gondii infections via cat vectors, present an important risk for pregnant women.

Plasmodium falciparum causes a severe form of human malaria and is responsible for nearly all malaria-specific mortality. Resistance of Plasmodium to antimalarial drugs is an increasingly serious problem in fighting the disease.

Ticks transmit babesiosis, and although this is primarily a disease of animals, humans are also infected with this parasite. There are over 100 species of Babesia, with Babesia microti and Babesia divergens the two most likely to cause human infection. Babesia microti is the organism responsible for a growing number of cases of infection especially in the northeast United States. Babesiosis is not only transmitted via tick bites, it can also be transmitted via blood transfusions, with documented cases of infection via this method.

There are numerous species of Eimeria, and an oralfecal route of transmission results in intestinal infection in cows, sheep, goats, pigs, ducks, chickens, turkeys, and rabbits, with the domestic chicken host to at least seven different species of Eimeria. Due to its widespread nature and its effects on the host animal, which may result in sub-optimal weight gain and reduced economic value, Eimeria is an economically important disease in the modern poultry production.

A subject may or may not exhibit symptoms of infection such as fever, swollen lymph glands, muscle aches, and pains. Methods to diagnose symptomatic and asymptomatic protozoan infection are known to those of ordinary skill in the art. Diagnostic methods include, for instance, but are not limited to, blood tests for antibodies to the protozoan parasite and other assays such as lymph assays for protozoan parasites. Scans by computerized tomography (CT scan) or magnetic resonance imaging (MRI scan) may also be used in the diagnosis of some types of protozoan infection, for example brain scans for Toxoplasma infection.

In some instances the inhibitors of the invention may be specially formulated for veterinary delivery. For instance, the inhibitors of the invention may be incorporated into animal feed or combined with other medications being delivered to an animal in order to treat the infection. Additionally, animal specific, i.e. cat-specific, antibiotics may be used on a cat in order to prevent a pregnant women who may be exposed to the cat from being exposed to an infection.

The therapeutic compounds described herein can be administered in combination with other therapeutic agents and such administration may be simultaneous or sequential. When the other therapeutic agents are administered simultaneously they can be administered in the same or separate formulations, but are administered at the same time. The administration of the other therapeutic agent, including anti-protozoan agents can also be temporally separated, meaning that the therapeutic agents are administered at a different time, either before or after, the administration of the therapeutics described herein. The separation in time between the administration of these compounds may be a matter of minutes or it may be longer.

Thus, in some instances, the invention also involves administering another anti-protozoan agents to a subject. Examples of existing or proposed anti-protozoan agents include Antiamoebic and antigiardiasis medicines: Diloxanide, Metronidazole; Antileishmaniasis medicines: Amphotericin B, Meglumine antimoniate, Pentamidine, Pentostam; Antimalarial medicines: Amodiaquine, Artemether, Artemether+lumefantrine, Artesunate, Chloroquine, Doxycycline, Mefloquine, Primaquine, Quinine, Sulfadoxine+pyrimethamine, Chloroquine, Doxycycline, Mefloquine, Proguanil, Malarone; Antipneumocytosis and antitoxoplasmosis medicines: Pentamidine, Pyrimethamine, Sulfamethoxazole+Trimethoprim; Antitrypanosomal medicines: African trypanosomiasis (Eflornithine, Melarsoprol, Pentamidine, Suramin sodium), American trypanosomiasis: (Benznidazole, Nifurtimox).

When used in combination with the therapies of the invention the dosages of known therapies may be reduced in some instances, to avoid side effects.

The active agents of the invention are administered to the subject in an effective amount for treating the subject. An “effective amount”, for instance, is an amount necessary or sufficient to realize a desired biologic effect. For instance an effective amount is that amount sufficient to prevent or inhibit protozoan growth or proliferation or alternatively an amount sufficient to induce apoptosis of a protozoan.

The effective amount of a compound of the invention in the treatment of a subject may vary depending upon the specific compound used, the mode of delivery of the compound, and whether it is used alone or in combination. The effective amount for any particular application can also vary depending on such factors as the type and/or degree of infection in a subject, the particular compound being administered for treatment, the size of the subject, or the severity of the disorder. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity in and of itself and yet is entirely effective to treat the particular subject.

Toxicity and efficacy of the protocols of the present invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays, animal studies and human studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

As used herein, the term treat, treated, or treating when used with respect to a disorder refers to a prophylactic treatment which increases the resistance of a subject to development of the disease or, in other words, decreases the likelihood that the subject will develop the disease as well as a treatment after the subject has developed the disease in order to fight the disease, prevent the disease from becoming worse, or slow the progression of the disease compared to in the absence of the therapy.

Multiple doses of the molecules of the invention are also contemplated. In some instances, when the molecules of the invention are administered with another therapeutic, for instance, a chemotherapeutic agent a sub-therapeutic dosage of either or both of the molecules may be used. A “sub-therapeutic dose” as used herein refers to a dosage which is less than that dosage which would produce a therapeutic result in the subject if administered in the absence of the other agent.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The compounds may be sterile or non-sterile.

The agent may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference). In a particular embodiment, intraperitoneal injection is contemplated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds of the invention may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients. In general, a pharmaceutical composition comprises the compound of the invention and a pharmaceutically-acceptable carrier. Pharmaceutically-acceptable carriers for nucleic acids, small molecules, peptides, monoclonal antibodies, and antibody fragments are well-known to those of ordinary skill in the art. As used herein, a pharmaceutically-acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In one embodiment, a kit comprises an inhibitor of protozoan tyrosyl-tRNA synthetase and instructions for administering the same. The kit may further comprise devices for administering the inhibitors, and/or other therapeutics or diagnostics related to the therapy.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

Computational techniques can be used to screen, identify, select, and design compounds capable of binding to the protozoan tyrosyl-tRNA synthetase or optionally the adenylate binding pocket thereof and functioning inhibitors. In particular, computational techniques can be used to identify or design ligands, such as agonists and/or antagonists, that associate with the protozoan tyrosyl-tRNA synthetase or the adenylate binding pocket thereof. Once identified and screened for biological activity, these agonists, antagonists, and combinations thereof, may be used therapeutically, for example, to inhibit protozoan tyrosyl-tRNA synthetase activity. Data stored in a machine-readable storage medium that is capable of displaying a graphical three-dimensional representation of the structure of the potential therapeutic compound or a structurally homologous molecule or molecular complex, as identified herein, or portions thereof may thus be advantageously used for drug discovery. The structure coordinates of the potential therapeutic compounds are used to generate a three-dimensional image that can be computationally fit to the three-dimensional image of the protozoan tyrosyl-tRNA synthetase or the adenylate binding pocket thereof. The three-dimensional molecular structure encoded by the data in the data storage medium can then be computationally evaluated for its ability to associate with the potential therapeutic compound. When the molecular structures encoded by the data is displayed in a graphical three-dimensional representation on a computer screen, the protein structure can also be visually inspected for potential association with the potential therapeutic compound. Additionally, programs are available for the “threading” of protozoan TyrRS sequences to existing known TyrRS structures for use in fitting, if the specific crystal structures of the individual protozoan TyrRS proteins are not in existence.

One embodiment of the method of drug design involves evaluating the potential association of a candidate therapeutic compound with the protozoan tyrosyl-tRNA synthetase or the adenylate binding pocket thereof. The method of drug design thus includes computationally evaluating the potential of a selected ligand to associate with any of the molecules or molecular complexes set forth above. This method includes the steps of: (a) employing computational means, for example, such as a programmable computer including the appropriate software known in the art or as disclosed herein, to perform a fitting operation between the potential therapeutic compound and the protozoan tyrosyl-tRNA synthetase or the adenylate binding pocket thereof and (b) analyzing the results of the fitting operation to quantify the association between the potential therapeutic compound and the protozoan tyrosyl-tRNA synthetase or the adenylate binding pocket thereof. Several methods can be used to screen potential therapeutic compounds for the ability to associate with the protozoan tyrosyl-tRNA synthetase or the adenylate binding pocket thereof. Selected potential therapeutic compounds may be positioned in a variety of orientations associating with the protozoan tyrosyl-tRNA synthetase or the adenylate binding pocket thereof. This may be accomplished using software such as QUANTA (Molecular Simulations, Inc., San Diego, Calif., USA.) and SYBYL (TRIPOS, St. Louis, Mo., USA), followed by energy minimization and molecular dynamics with standard molecular mechanics force fields, such as CHARMM (Molecular Simulations, Inc., San Diego, Calif., USA) and AMBER (P. A. Kollman, University of California at San Francisco, San Francisco, Calif., USA).

Any of the biological or biochemical functions mediated by the protozoan tyrosyl-tRNA synthetase or the adenylate binding pocket thereof can be used as an endpoint assay to identify an agent that modulates protozoan tyrosyl-tRNA synthetase activity (a putative therapeutic compound). The assays may include all of the biochemical or biochemical/biological events described herein, in the references-cited herein, incorporated by reference for these endpoint assay targets, and other functions known to those of ordinary skill in the art or that can be readily identified. Compounds can be identified through cellular assays. Cellular assays may involve, for instance, expressing the protozoan tyrosyl-tRNA synthetase or the adenylate binding pocket thereof in cells and testing a variety of compounds for their ability to bind to the expressed peptide. The assay may be performed with labeled compounds, facilitating identification of the compound that binds. In another embodiment a biological readout can be used to identify a putative therapeutic compound. Biological assays will allow for the identification of both agonists and antagonists or inhibitors. Competition binding assays may also be used to discover compounds that interact with the protozoan tyrosyl-tRNA synthetase or the adenylate binding pocket thereof (e.g. binding partners and/or ligands). Thus, a compound is exposed to the protozoan tyrosyl-tRNA synthetase or the adenylate binding pocket thereof under conditions that allow the compound to bind or to otherwise interact with the polypeptide. A peptide or antibody or fragment thereof against the protozoan tyrosyl-tRNA synthetase or the adenylate binding pocket thereof may be added to the mixture. If the test compound interacts with the protozoan tyrosyl-tRNA synthetase or the adenylate binding pocket thereof, it decreases the amount of peptide or antibody that can bind to the protozoan tyrosyl-tRNA synthetase or the adenylate binding pocket thereof. To perform cell free drug screening assays, it is sometimes desirable to immobilize the protozoan tyrosyl-tRNA synthetase or the adenylate binding pocket thereof, or its target molecule to facilitate separation of complexes from uncomplexed forms of one or both of the proteins, as well as to accommodate automation of the assay. Agents that modulate the protozoan tyrosyl-tRNA synthetase or the adenylate binding pocket thereof can be identified using one or more of the above assays, alone or in combination. It is generally preferable to use a cell-based or cell free system first and then confirm activity in an animal or other model system. Such model systems are well known in the art and can readily be employed in this context.

Some additional sequences for tyrosyl tRNA synthetases are included below.

Cryptosporidium parvum (GI: 323509769):
(SEQ ID NO. 2)
MVILVKYSKT SNIGKVVSLL VNYLLKQNTE LKIEESIIDE
NSTNEKNINQ VVLIKDKENC NDQLESIVDI LIYLGSFGKN
LLGKDSSLIN ESKEIIEKLI KINFNFSKGT EMSEFNEMIK
GKSFFLGRSL TIVDLVIYIS LYSYFESLKD QNQDYIPEYP
NLSTFFEQIQ IVTLIRQSCP EGINYLDLPF FKKKLVKKEK
KNTGEHGNNS NNNKQEERPL DDPTRIALRV GRILSVEKHP
TADKLYLEKI DVGEDEPRTI LSGLVGVYDL TQKINKLVVI
VSNLKPRAMR GITSNGMLLC ASSAPSGDNN NNNSQNQSSK
EYCEPVSVPK DAKVGELIFY NDFKGEPDVV LNTKTGKDPF
AAVQPNYNVS SDLLCRFKES TMMTSAGPVF VENKNLIHGK
LS
Human (GI: 119627912 or 2665519):
(SEQ ID NO 3)
MGDAPSPEEK LHLITRNLQE VLGEEKLKEI LKERELKIYW
GTATTGKPHV AYFVPMSKIA DFLKAGCEVT ILFADLHAYL
DNMKAPWELL ELRVSYYENV IKAMLESIGV PLEKLKFIKG
TDYQLSKEYT LDVYRLSSVV TQHDSKKAGA EVVKQVEHPL
LSGLLYPGLQ ALDEEYLKVD AQFGGIDQRK IFTFAEKYLP
ALGYSKRVHL MNPMVPGLTG SKMSSSEEES KIDLLDRKED
VKKKLKKAFC EPGNVENNGV LSFIKHVLFP LKSEFVILRD
EKWGGNKTYT AYVDLEKDFA AEVVHPGDLK NSVEVALNKL
LDPIREKFNT PALKKLASAA YPDPSKQKPM AKGPAKNSEP
EEVIPSRLDI RVGKIITVEK HPDADSLYVE KIDVGEAEPR
TVVSGLVQFV PKEELQDRLV VVLCNLKPQK MRGVESQGML
LCASIEGINR QVEPLDPPAG SAPGEHVFVK GYEKGQPDEE
LKPKKKVFEK LQADFKISEE CIAQWKQTNF MTKLGSISCK
SLKGGNIS
Escherichia coli str. K-12 (GI: 16129595):
(SEQ ID NO. 4)
MASSNLIKQL QERGLVAQVT DEEALAERLA QGPIALYCGF
DPTADSLHLG HLVPLLCLKR FQQAGHKPVA LVGGATGLIG
DPSFKAAERK LNTEETVQEW VDKIRKQVAP FLDFDCGENS
AIAANNYDWF GNMNVLTFLR DIGKHFSVNQ MINKEAVKQR
LNREDQGISF TEFSYNLLQG YDFACLNKQY GVVLQIGGSD
QWGNITSGID LTRRLHQNQV FGLTVPLITK ADGTKFGKTE
GGAVWLDPKK TSPYKFYQFW INTADADVYR FLKFFTFMSI
EEINALEEED KNSGKAPRAQ YVLAEQVTRL VHGEEGLQAA
KRITECLFSG SLSALSEADF EQLAQDGVPM VEMEKGADLM
QALVDSELQP SRGQARKTIA SNAITINGEK QSDPEYFFKE
EDRLFGRFTL LRRGKKNYCL ICWK.
Staphylococcus aureus (GI: 16975033):
(SEQ ID NO. 5)
MTNVLIEDLK WRGLIYQQTD EQGIEDLLNK EQVTLYCGAD
PTADSLHIGH LLPFLTLRRF QEHGHRPIVL IGGGTGMIGD
PSGKSEERVL QTEEQVDKNI EGISKQMHNI FEFGTDHGAV
LVNNRDWLGQ ISLISFLRDY GKHVGVNYML GKDSIQSRLE
HGISYTEFTY TILQAIDFGH LNRELNCKIQ VGGSDQWGNI
TSGIELMRRM YGQTDAYGLT IPLVTKSDGK KFGKSESGAV
WLDAEKTSPY EFYQFWINQS DEDVIKFLKY FTFLGKEEID
RLEQSKNEAP HLREAQKTLA EEVTKFIHGE DALNDAIRIS
QALFSGDLKS LSAKELKDGF KDVPQVTLSN DTTNIVEVLI
ETGISPSKRQ AREDVNNGAI YINGERQQDV NYALAPEDKI
DGEFTIIRRG KKKYFMVNYQ.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

When a range of values is listed, it is intended to encompass each value and sub range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tertbutyl (C₄), secbutyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

“Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, CF₂Cl, and the like.

As used herein, “alkenyl” refers to a radical of a straight-chain or branched hydrocarbon group having from 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “alkynyl” refers to a radical of a straight-chain or branched hydrocarbon group having from 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, heteroalkyl, heteroalkenyl, and heteroalkynyl refers to an alkyl, alkenyl, and alkynyl moiety, as defined herein, wherein 1, 2, 3, or 4 carbons of the parent chain are replaced with oxygen, nitrogen, or sulfur atoms.

As used herein, “carbocyclyl” refers to a radical of a nonaromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” refers to a radical of a 3 to 14-membered non aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered nonaromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered nonaromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered nonaromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2b]pyrrole, indolinyl, phthalimidyl, naphthalimidyl, chromanyl, chromenyl, 1H-benzo[e][1,4]diazepinyl, 1,4,5,7-tetrahydropyrano[3,4b]pyrrolyl, 5,6-dihydro-4H-furo[3,2b]pyrrolyl, 6,7-dihydro-5H-furo[3,2b]pyranyl, 5,7-dihydro-4H-thieno[2,3c]pyranyl, 2,3-dihydro-1H-pyrrolo[2,3b]pyridinyl, 2,3-dihydrofuro[2,3b]pyridinyl, 4,5,6,7-tetrahydro-1H-pyrrolo[2,3b]pyridinyl, 4,5,6,7-tetrahydrofuro[3,2c]pyridinyl, 4,5,6,7-tetrahydrothieno[3,2b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14 it electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group, as defined herein, substituted by an aryl group, as defined herein, wherein the point of attachment is on the alkyl moiety.

As used herein, “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group, as defined herein, substituted by a heteroaryl group, as defined herein, wherein the point of attachment is on the alkyl moiety.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, alkenylene is the divalent moiety of alkenyl, and alkynylene is the divalent moiety of alkynyl, each as defined herein.

Alkyl, alkenyl, alkynyl, heteroalkyl, heteroalkenyl, heteroalkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₁₋₁₀ heteroalkyl, C₁₋₁₀ heteroalkenyl, C₂₋₁₀heteroalkynyl, C₃₋₁₄ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₁₋₁₀ heteroalkyl, C₁₋₁₀ heteroalkenyl, C₂₋₁₀heteroalkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R)₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R)₂, —P(═O)(NR^{cc})₂, C₁₋₁₀alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀heteroalkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{CC} is, independently, selected from hydrogen, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀heteroalkynyl, C₃₋₁₀ carbocyclyl, 3-14 membered heterocyclyl, C₆₋₁₄ aryl, and 5-14 membered heteroaryl, or two R^{CC} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₁₋₆ heteroalkyl, C₂₋₆ heteroalkenyl, C₂₋₆heteroalkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁ perhaloalkyl, C₂₋₆ alkenyl, C₂₋₆ alkynyl, C₁₋₆ heteroalkyl, C₂₋₆ heteroalkenyl, C₂₋₆heteroalkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₁₋₆ heteroalkyl, C₂₋₆ heteroalkenyl, C₂₋₆heteroalkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₅ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₅ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₅ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁ perhaloalkyl, C₂₋₆ alkenyl, C₂₋₆ alkynyl, C₁₋₆heteroalkyl, C₂₋₆ heteroalkenyl, C₂₋₆heteroalkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-14 membered heterocyclyl, C₆₋₁₄ aryl, and 5-14 membered heteroaryl, or two R^{CC} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{CC} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₁₋₁₀ heteroalkyl, C₂₋₁₀ heteroalkenyl, C₂₋₁₀ heteroalkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{CC} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3(p-hydroxyphenyl)propanamide, 3(o-nitrophenyl)propanamide, 2-methyl-2(o-nitrophenoxy)propanamide, 2-methyl-2(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′ methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)acyl derivative, N′ p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2 picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O₂N(R^{bb})₂, and —P(═O)(NR^{bb})₂, wherein R^{aa}, R^{bb}, and R^{CC} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2 picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, tbutyldimethylsilyl (TBDMS), t-butyldiphenylsilyl (TBDPS), tribenzylsilyl, tripxylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2(trimethylsilyl)ethyl carbonate (TMSEC), 2-(phenylsulfonyl)ethyl carbonate (Psec), 2(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl pmethoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on an sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{CC} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and claims. The invention is not intended to be limited in any manner by the above exemplary listing of substituents.

As used herein, the term “salt” refers to any and all salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “prodrug” means a compound that can react under biological conditions (e.g., in vitro or in vivo enzymatic conditions) to provide a pharmacologically active compound. In certain cases, a prodrug has improved physical and/or delivery properties over the parent compound. Prodrugs are typically designed to enhance pharmacologically, pharmaceutically and/or pharmacokinetically based properties associated with the parent compound. The advantage of a prodrug can lie in its physical properties, such as enhanced water solubility compared to the parent compound, enhanced transport across a cell membrane, and/or enhanced drug stability for long-term storage.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for treating protozoan disease, comprising:
administering to a subject having a protozoan disease an inhibitor of protozoan tyrosyl-tRNA synthetase in an effective amount to treat the protozoan disease, wherein the inhibitor of protozoan tyrosyl-tRNA synthetase does not inhibit mammalian tyrosyl-tRNA synthetase.

2. The method of claim 1, wherein the inhibitor of protozoan tyrosyl-tRNA synthetase specifically binds to an adenylate binding pocket of protozoan tyrosyl-tRNA synthetase.

3. The method of claim 2, wherein the adenylate binding pocket is a region of the protozoan tyrosyl-tRNA synthetase corresponding to F70, Q80, K84, C85 and F127 of SEQ ID NO. 1.

4. The method of claim 2, wherein the adenylate binding pocket is a region of the protozoan tyrosyl-tRNA synthetase corresponding to F70, Q80, and F127 of SEQ ID NO. 1.

5. The method of claim 1, wherein the inhibitor of protozoan tyrosyl-tRNA synthetase is also an inhibitor of bacterial tyrosyl-tRNA synthetase.

6. The method of claim 1, wherein the inhibitor of protozoan tyrosyl-tRNA synthetase is not an inhibitor of bacterial tyrosyl-tRNA synthetase.

7. The method of claim 1, wherein the protozoan disease is selected from the group consisting of malaria, toxoplasma, leishmaniasis, giardiasis, trypanosomiasis (sleeping sickness, Chagas disease), amoebic dysentery, and trichomoniasis.

8. The method of claim 1, wherein the inhibitor of protozoan tyrosyl-tRNA synthetase is a compound of Formula (I) 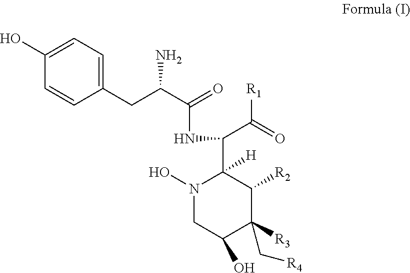
or a salt thereof;
wherein:
each instance of R1, R2, R3, and R4 is, independently, hydrogen, hydroxyl, substituted or unsubstituted OR′, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R′ is substituted or unsubstituted alkyl, substituted or unsubstituted alkenyl, substituted or unsubstituted alkynyl.

9. The method of claim 1, wherein the inhibitor of protozoan tyrosyl-tRNA synthetase is SB 219383.

10. The method of claim 1, wherein the inhibitor of protozoan tyrosyl-tRNA synthetase is SB 239629.

11. The method of claim 1, wherein the inhibitor of protozoan tyrosyl-tRNA synthetase is SB 243545.

12. The method of claim 1, wherein the inhibitor of protozoan tyrosyl-tRNA synthetase is SB 284485.

13. A composition, comprising:
an inhibitor of protozoan tyrosyl-tRNA synthetase, wherein the inhibitor of protozoan tyrosyl-tRNA synthetase does not inhibit mammalian or bacterial tyrosyl-tRNA synthetase.

14. A method of selecting or designing a compound that interacts with a protozoan tyrosyl-tRNA synthetase and modulates protozoan tyrosyl-tRNA synthetase activity, the method comprising the step of assessing the stereochemical complementarity between the compound and a topographic region of protozoan tyrosyl-tRNA synthetase, wherein the topographic region of the protozoan tyrosyl-tRNA synthetase is characterized by at least a portion of the amino acids positioned at an adenylate binding pocket of the protozoan tyrosyl-tRNA synthetase.

15. The method of claim 14, wherein the adenylate binding pocket is a region of the protozoan tyrosyl-tRNA synthetase corresponding to F70, Q80, K84, C85 and F127 of SEQ ID NO. 1.

16. The method of claim 14, wherein the adenylate binding pocket is a region of the protozoan tyrosyl-tRNA synthetase corresponding to F70, Q80, and F127 of SEQ ID NO. 1.

17. (canceled)

18. The method of claim 1, wherein the inhibitor of protozoan tyrosyl-tRNA synthetase is a compound of Formula (I) 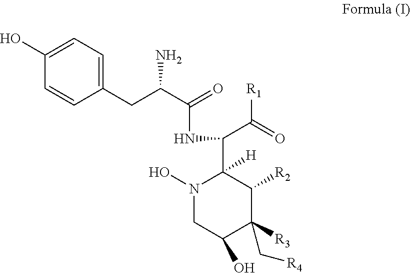
or a salt thereof;
wherein:
each instance of R1, R2, R3, and R4 is, independently, hydrogen, hydroxyl, substituted or unsubstituted OR′, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R′ is substituted or unsubstituted alkyl, substituted or unsubstituted alkenyl, substituted or unsubstituted alkynyl.

19. The method of claim 1, wherein the inhibitor of protozoan tyrosyl-tRNA synthetase is SB 219383.

20. The method of claim 1, wherein the inhibitor of protozoan tyrosyl-tRNA synthetase is SB 239629.

21. The method of claim 1, wherein the inhibitor of protozoan tyrosyl-tRNA synthetase is SB 243545.

22. The method of claim 1, wherein the inhibitor of protozoan tyrosyl-tRNA synthetase is SB 284485.

23. A compound of the formula (I) or (II): 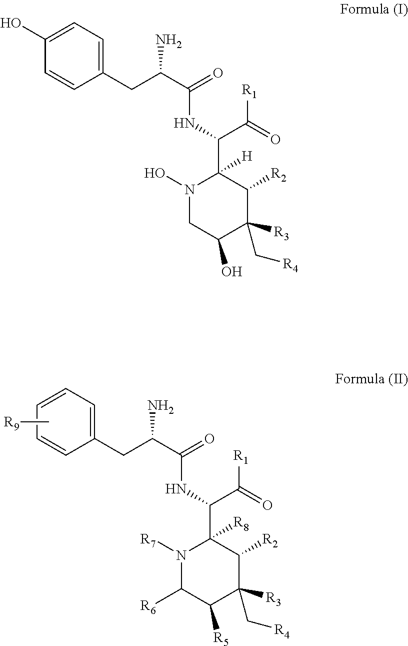
or a salt thereof;
wherein:
each instance of R1, R2, R3, and R4 is, independently, hydrogen, hydroxyl, substituted or unsubstituted OR′, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R′ is substituted or unsubstituted alkyl, substituted or unsubstituted alkenyl, substituted or unsubstituted alkynyl;
wherein each instance of R5, R6, R7, R8 and R9 is, independently, hydrogen, halogen, substituted or unsubstituted alkyl, substituted or unsubstituted alkenyl, substituted or unsubstituted alkynyl; and
provided that the compound of Formula (I) is not a compound wherein R1 is a butyl ester or OH and R2, R3, and R4 are all OH,
provided that the compound of Formula (II) is not a compound wherein R1 is a butyl ester or OH, and R2, R3, R4, R5, and R7 are all OH, R9 are all H and one OH, and R6 and R8 are both H.

24. The compound of claim 23, wherein the 3′ and 4′ hydroxyl groups on the ring are modified such that the compound has a strong reaction with an amino acid in the adenylate binding pocket corresponding to Cys residue at position 85 of SEQ ID NO.1.


SEQUENCE LISTING (13 sequences)

SEQ ID NO: 1 - protein, 380 aa, Plasmodium vivax
MEGEGVKREELEGATGEAAPQEAASQEATPQEDVSAKLADILSVASECIQPEELKARLLLKRRLVCYDGFEPSGRMHIAQGLLKCQIVNKLTSNGCRFIFWIADWFAQLNNKMSGDLKKIKKVGMYFIEVWKSCGMNMQNVEFLWASEEINKKPNEYWSLVIDISKSFNINRIKRCLKIMGRSEGEENYCSQILYPCMQCADIFFLNVDICQLGIDQRKVNMLAREYCEIKKMKKKPIILSHQMLPGLLEGQEKMSKSDENSAIFMDDSEADVNRKIKKGYCPPGVIESNPIFAYARSIVFPHYNEFALQRKEKNGGNKTYATIAELEADYLSGALHPLDLKDNVAIYLNKMLQPVRDHFQNDAAAKSLLSEIKKYKVTK

SEQ ID NO: 2 - protein, 402 aa, Cryptosporidium parvum
MVILVKYSKTSNIGKVVSLLVNYLLKQNTELKIEESIIDENSTNEKNINQVVLIKDKENCNDQLESIVDILIYLGSFGKNLLGKDSSLINESKEIIEKLIKINFNFSKGTEMSEFNEMIKGKSFFLGRSLTIVDLVIYISLYSYFESLKDQNQDYIPEYPNLSTFFEQIQIVTLIRQSCPEGINYLDLPFFKKKLVKKEKKNTGEHGNNSNNNKQEERPLDDPTRIALRVGRILSVEKHPTADKLYLEKIDVGEDEPRTILSGLVGVYDLTQKINKLVVIVSNLKPRAMRGITSNGMLLCASSAPSGDNNNNNSQNQSSKEYCEPVSVPKDAKVGELIFYNDFKGEPDVVLNTKTGKDPFAAVQPNYNVSSDLLCRFKESTMMTSAGPVFVENKNLIHGKLS

SEQ ID NO: 3 - protein, 528 aa, Homo sapiens
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 4 - protein, 424 aa, Escherichia coli
MASSNLIKQLQERGLVAQVTDEEALAERLAQGPIALYCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYDFACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 5 - protein, 420 aa, Staphylococcus aureus
MTNVLIEDLKWRGLIYQQTDEQGIEDLLNKEQVTLYCGADPTADSLHIGHLLPFLTLRRFQEHGHRPIVLIGGGTGMIGDPSGKSEERVLQTEEQVDKNIEGISKQMHNIFEFGTDHGAVLVNNRDWLGQISLISFLRDYGKHVGVNYMLGKDSIQSRLEHGISYTEFTYTILQAIDFGHLNRELNCKIQVGGSDQWGNITSGIELMRRMYGQTDAYGLTIPLVTKSDGKKFGKSESGAVWLDAEKTSPYEFYQFWINQSDEDVIKFLKYFTFLGKEEIDRLEQSKNEAPHLREAQKTLAEEVTKFIHGEDALNDAIRISQALFSGDLKSLSAKELKDGFKDVPQVTLSNDTTNIVEVLIETGISPSKRQAREDVNNGAIYINGERQQDVNYALAPEDKIDGEFTIIRRGKKKYFMVNYQ

SEQ ID NO: 6 - protein, 63 aa, Giardia
DGFEPSGRIHIAQGIAKAINVNRLLRSGIDCVFYVADWFAALNNKCDAELSKIQILGRYFVEV

SEQ ID NO: 7 - protein, 63 aa, Entamoeba
NGFEPSGRIHIAQAILTVLNANKIHECGGLYKIYIADWFAQLNHKMGGDLSKIQVLGKYFIEV

SEQ ID NO: 8 - protein, 63 aa, Trichomoniasis
NGFEPSGRMHIAQALVTAINCNAITKAGGTMVLYIADLFAQLNHKMGGDMKKIHDVGRYFVEV

SEQ ID NO: 9 - protein, 63 aa, Dictyostelium
DGFEPSGRMHIAQGILKSINVNRMTKAGCTFIFWVADWFAMLNNKMNGDLNKIRKVGQYMIEI

SEQ ID NO: 10 - protein, 63 aa, Leishmania
DGFEPSGRMHIAQGIFKAVNVNKCTAAGCEFVFWVADWFALMNDKVGGELEKIRIVGRYLIEV

SEQ ID NO: 11 - protein, 63 aa, Plasmodium
DGFEPSGRMHIAQGLLKCQIVNKLTSNGCRFIFWIADWFAQLNNKMSGDLKKIKKVGMYFIEV

SEQ ID NO: 12 - protein, 61 aa, Homo sapiens
WGTATTGKPHVAYFVPMSKIADFLKAGCEVTILFADLHAYLDNMKAPWELLELRVSYYENV

SEQ ID NO: 13 - protein, 70 aa, Staphylococcus aureus
CGADPTADSLHIGHLLPFLTLRRFQEHGHRPIVLIGGGTGMIGDPSGKSEERVLQTEEQVDKNIEGISKQ